SEQUENCE LISTING (6 sequences)

SEQ ID NO: 1 - protein, 10 aa, synthetic construct
GYTFTSCVMN

SEQ ID NO: 2 - protein, 17 aa, synthetic construct
YINPYNDGTKYNEKFKG

SEQ ID NO: 3 - protein, 10 aa, synthetic construct
EGFYYGNFDN

SEQ ID NO: 4 - protein, 11 aa, synthetic construct
RASENIYSYLT

SEQ ID NO: 5 - protein, 7 aa, synthetic construct
NAKTLPE

SEQ ID NO: 6 - protein, 9 aa, synthetic construct
QHHYGTPYT

(19) United States
(12) Patent Application Publication
(10) Pub. No.: US 2025/0034278 A1
(43) Pub. Date: Jan. 30, 2025

(54) USE OF ALPHA-ENOLASE ANTAGONIST FOR TREATING ANGIOGENESIS-RELATED DISEASES

(71) Applicant: HUNILIFE BIOTECHNOLOGY, INC., Taipei City (TW)

(72) Inventors: Ta-Tung YUAN, Taipei City (TW); WEI-CHING HUANG, Taipei City (TW)

(21) Appl. No.: 18/716,249

(22) PCT Filed: Jan. 17, 2023
(86) PCT No.: PCT/CN2023/072636

§ 371 Date: Jun. 4, 2024

Related U.S. Application Data
(60) Provisional application No. 63/303,421, filed on Jan. 26, 2022.

(51) Int. Cl. C07K 16/40; A61K 39/00; A61P 35/00

(52) U.S. Cl. CPC C07K 16/40; A61P 35/00; C12Y 402/01011; A61K 2039/505; C07K 2317/24; C07K 2317/76

(57) ABSTRACT

Provided herein are methods for treating an angiogenesis-related disease comprising administration to a subject in need thereof an effective amount of alpha-enolase (enolase-1. ENO-1) antagonist.

### CROSS-REFERENCE TO RELATED APPLICATION

This application claims the benefit of U.S. Provisional Application No. 63/303,421, filed Jan. 26, 2022, which is hereby incorporated by reference herein for all purposes.

### BACKGROUND OF INVENTION

### Field of the Invention

The present disclosure relates to the medical field of angiogenesis. More specifically, the disclosure relates to the use of alpha-enolase (enolase-1, ENO-1) antagonist for inhibition of angiogenesis and thereof the treatment and/or prevention of angiogenesis-related diseases, particularly age-related macular degeneration, and diabetic retinopathy.

### Background Art

Angiogenesis is the formation of new capillaries from pre-existing blood vessels. When dysregulated, the formation of new blood vessels contributes to numerous malignant, ischemic, inflammatory, infectious, and immune disorders. These angiogenesis-related diseases include but are not limited to cancers, vascular neoplasms & malformations (angiofibroma, arteriovenous malformations, hemangiomatosis), cardiovascular, pulmonary, and central nerve system diseases (atherosclerosis, vascular adhesions, vascular dementia, restenosis/reperfusion injury, pulmonary fibrosis, Alzheimer's diseases), ocular disorders (corneal neovascular diseases, retinal neovascular diseases, retinal angiomatous proliferation, polypoidal choroidal vasculopathy, neovascular age-related macular degeneration, diabetic retinopathy, diabetic macular edema, neovascular glaucoma, ischemic retinopathy, retrolental fibroplasia, or retinopathy of prematurity), or chronic inflammatory diseases (Crohn's disease, diabetes, psoriasis, pyogenic granuloma, periodontitis, rheumatoid arthritis, or systemic sclerosis).

Angiogenesis is mediated by a complex multistep process comprising a series of cellular events that lead to neovascularization. Angiogenic factors, such as vascular endothelial growth factors (VEGF), cause increased proliferation and migration of vascular endothelial cells. Invasion of the endothelial cells into the stroma of neighboring tissue requires proteolysis of basement membrane, in which the cooperative activity of the plasminogen activator system and matrix metalloproteinases. VEGF inhibitors can, therefore, be of considerable value in the treatment of angiogenesis-dependent diseases. For example, anti-VEGF therapies could induce tumor regression or prevent the blindness associated with neovascular age-related macular degeneration or proliferative diabetic retinopathy. However, primary and secondary non-responsiveness occur when using anti-VEGF therapies. New anti-angiogenesis strategies are still needed.

Tissue hypoxia is sensed by the hypoxia-inducible factor-1α (HIF-1α) which impact angiogenesis in multiple ways. HIF-la serves as a main transcriptional factor activating transcription of genes encoding glycolytic enzymes, VEGF, and other proteins that are important for maintaining oxygen homeostasis. Glycolysis, via HIF-1α, controls vascular endothelia cells proliferation and migration.

ENO-1 is a multiple functional protein, which was first found as a cytosolic enzyme of the glycolysis pathways. Adenosine triphosphate (ATP) is involved in the growth and proliferation of endothelial cells of the vessels. In the context of hypoxia, glycolysis is a compensatory adaptation process of energy metabolism to obtain the necessary energy for life activities. ENO-1 is demonstrated to be up-regulated in the hypoxic condition under the control of HIF-1α. The activation ENO-1-mediated glycolytic pathway, improves the energy imbalance of hypoxic cells, promotes the transcription, proliferation and inhibits cell apoptosis, leading to the promotion of angiogenesis.

ENO-1 is also found to express on the cell surfaces as a plasminogen receptor. It is known that the upregulation of plasminogen receptor proteins can induce a cascade response of the urokinase plasminogen activation system and results in extracellular matrix degradation, which are required in angiogenesis.

### SUMMARY OF INVENTION

The invention relates to an alpha-enolase (enolase-1, ENO-1) antagonist targeting ENO-1 and a use thereof, wherein the ENO-1 antagonist has a binding capacity to ENO-1, e.g. human ENO-1 antibody, as an antigen binding structural domain so as to neutralizing the biological effects of the ENO-1. The ENO-1 antagonist can bind to free ENO-1 protein and ENO-1 protein on the surface of a cell and has an important application prospect in the treatment of angiogenesis-related diseases.

In accordance with certain embodiments of the disclosure, the angiogenesis-related diseases or disorders may be any condition arising from aberrant activation or expression of ENO-1 protein.

In accordance with certain embodiments of the disclosure, the angiogenesis-related diseases or disorders may include (1) ocular neovascular disease (such as, retinal neovascular diseases, neovascular age-related macular degeneration, diabetic retinopathy, and retinopathy of prematurity), a disease characterized by invasion of new blood vessels into the retina or cornea, leading to irreversible visual impermanent; (2) rheumatoid arthritis and osteoarthritis; the cells within subchondral spaces and vascular channels and the chondrocytes secrete various growth factor that induce angiogenesis, which may result in the pathogenesis of synovitis and the subsequent destruction of the articular cartilage; (3) inflammation and inflammatory disease; during inflammation, the inflammatory cytokines (such as cyclooxygenase 2,prostaglandin E2, and thromboxane A2), the inflammatory cells (such as macrophage, T cell, and mast cell), and the hypoxia-inducible factor (HIF) elicit the expression of pro-angiogenic factors that cause angiogenesis; the newly formed vasculature in turn enhance the infiltration of the inflammatory cells, which further deteriorate the inflammation; (4) hereditary diseases, such as Osler-Weber-Rendu disease or hereditary hemorrhagic telangiectasia, are related to the disorder of angiogenesis, in which a multisystemic angiodysplasia is responsible for severe hemorrhage; and (5) tumor formation and metastasis; tumor cells secret various pro-angiogenic factors that promote the formation of new blood vessels via angiogenesis process; the new blood vessels are important in tumor growth by providing tumor cells adequate nutrients and oxygen, and are corrected with tumor metastasis by providing a route for tumor migration and invasion.

In accordance with certain embodiments of the disclosure, the administering is by oral, parenteral, buccal, vaginal, rectal, inhalation, insufflation, sublingual, intramuscular, subcutaneous, topical, intranasal, intraperitoneal, intrathoracic, intravenous, epidural, intrathecal, intracerebroventricular, intraocular, or intravitreal route.

In accordance with certain embodiments of the disclosure, the subject is a mammal. In a preferred embodiment the subject is human.

In accordance with certain embodiments of the disclosure, the ENO-1 antagonist can be a nucleic acid, e.g. DNA or RNA, designed to be delivered into cells and expressed as intracellular protein of peptide. For example, the ENO-1 antagonist can be a nucleic acid that can be transcribed and translated into, for instance, an anti-ENO-1 antibody or the binding fragment thereof. In addition, the nucleic acid can be with or without a secretion signal peptide so that the proteins or the peptides transcribed and translated from the nucleic acid can be secreted, non-secreted or a combination thereof. In accordance with certain embodiments of the disclosure, the nucleic acid can be delivered to the subject via any known methods or medium, e.g. viral vector, polymer, or liposome. In accordance with certain embodiments of the disclosure, the nucleic acid may be substituted with known modified nucleotides, e.g. Pseudo UTP, 1-Me pseudo UTP, 5-Methoxy UTP, N1-Ethyl pseudo UTP, 5-Methyl CTP or N4-Acetyl-CTP, to enhance the expression efficiency.

Compared with the prior art, the disclosed inventions have the following beneficial effects. According to the present disclosure, in vitro experiments demonstrate the anti-angiogenesis efficacy of the ENO-1 antagonist, e.g. ENO-1 monoclonal antibody. That is, the ENO-1 antagonist is a potential therapy for the angiogenesis-related diseases, e.g. neovascular age-related macular degeneration, via novel mechanism of inhibition on plasmin activation and glycolysis reprogramming.

One skilled in the art would appreciate that the pharmaceutically effective amount depends on many factors, such as patient conditions, age, disease states, routes of administration, etc., and that such effective amount may be determined based on these factors in routine practice without undue experimentation.

Other aspect of the disclosed invention will become apparent with the following description.

### BRIEF DESCRIPTION OF THE DRAWINGS

FIG. 1 is a set of photographs showing the increased surface ENO-1 expression in VEGF-A-stimulated HUVEC.

FIG. 2 shows reduced plasmin activity and cell migration of VEGF-A-stimulated HUVEC treated with ENO-1 antibody.

FIG. 3 shows reduced tube formation of VEGF-A-stimulated HUVEC treated with ENO-1 antibody.

FIG. 4 shows ENO-1 antibody reduced vessel sprouting in rat aortic ring model of angiogenesis.

FIG. 5 shows ENO-1 antibody reduced angiogenesis in vivo in Matrigel plug assay.

FIG. 6 shows ENO-1 antibody reduced angiogenesis in vivo in a prostate

cancer subcutaneous xenograft model.

### DETAILED DESCRIPTION

General Definitions

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

The terms “ENO-1 antagonist” refers to the molecule with a binding capacity to ENO-1 as an antigen binding structural domain so as to neutralize the biological effects of the ENO-1.

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The antibodies can be full-length or can comprise a fragment (or fragments) of the antibody having an antigen-binding portion, including, but not limited to, Fab, F(ab′)₂, Fab′, F(ab)′, Fv, single chain Fv(scFv), bivalent scFv(bi-scFv), trivalent scFv (tri-scFv), Fd, dAb fragment (e.g., Ward et al, Nature, 341:544-546(1989)), an CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention. Bird et al. Science, 1988,242:423-426. Huston et al, Proc. Natl. Acad. Sci. USA, 1988, 85:5879-5883.

As used herein, “treatment” or “treating” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the present disclosure are used to delay development of a disease or disorder.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice, and rats. In certain embodiments, the vertebrate is a human.

An “effective amount” refers to an amount effective, at dosages and for periods of time necessary, to achieve the desired therapeutic or prophylactic result. However, “effective amount” of a substance/molecule of the present disclosure may vary according to factors such as the disease state, age, sex, and weight of the individual, and the ability of the substance/molecule, to elicit a desired response in the individual. An effective amount is also one in which any toxic or detrimental effects of the substance/molecule are outweighed by the therapeutically beneficial effects. In one embodiment the effective amount of the anti-ENO-1 antibody ranges from 1-1000 mg/kg, preferably 5-100 mg/kg, more preferably 10-50 mg/kg, e.g. 5, 10, 15, 20, 30, 40, 50, 60, 70, 80, 90 or 100 mg/kg.

The term “angiogenesis” as used herein refers to a biological process that involves the sprouting of new blood vessels from pre-existing ones and plays a crucial role in disease development and progression [Folkman J. N Engl J Med 1995,333:1757-1763]. Angiogenesis is a complex process in which endothelial cells serve as a building block for blood vessel expansion. It is regulated through a fine balance between pro-angiogenic and anti-angiogenic molecules.

The control of angiogenesis is altered in certain diseases. Many such diseases involve pathological angiogenesis (i.e., inappropriate, excessive or undesired blood vessel formation), which supports the disease state and, in many instances, contributes to the cellular and tissue damage associated with such diseases. Angiogenesis-related diseases (i.e., those involving pathological angiogenesis) are myriad and varied. They include, but are not limited to, various forms of tumors, chronic inflammatory diseases, and neovascularization diseases. In an embodiment, angiogenesis-related diseases may include retinal neovascular diseases, neovascular age-related macular degeneration, diabetic retinopathy, retinopathy of prematurity, or caners.

For a general discussion of the role of angiogenesis in angiogenesis-related diseases see the following references: Moses et al., 1991, BioTechol. 9:630-633; Leek et al., 1994, J. Leuko. Biol. 56:423-435; and Beck et al., 1997, FASEB J. 11:365-373.

In an embodiment, the ENO-1 antagonist may be administered with anti-angiogenic agents for the treatment of an angiogenesis-related disease. Many anti-angiogenic agents have been isolated or developed. They include cartilage-derived factors (Moses et al., 1990, 248:1408-1410; Oikawa et al., 1990, Cancer Lett. 51:181-186); angiostatic steroids (Folkman et al., 1983, Science 221:719-725; Crum et al., 1985, Science 230:1375-1378; Oikawa et al., 1988, Cancer Lett. 43:85-92); and angiostatic vitamin D analogs (Oikawa et al., 1989, Cancer Lett. 48:157-162; Oikawa et al., 1990, Eur. J. Pharmacol. 178:247-50) angiostatin (O'Reilly et al., 1994, Cell 79:315-328), endostatin (O′Reilly et al., 1997, Cell 88:277-285), and verostatin (Pike et al. 1998, J. Exp. Med. 88:2309-2356). Consistent with the idea that pathological angiogenesis underlies angiogenesis-related diseases, many anti-angiogenic agents have been demonstrated to have beneficial therapeutic activity against such diseases.

The medicaments of the present disclosure may be applied locally or systemically. The medicaments of the present disclosure may also be supplied in combinations or with cofactors. Medicaments of the present disclosure may be administered in an amount sufficient to restore normal levels, if the medicament of the present disclosure is normally present in the target location, or they may be administered in an amount to raise levels above normal levels in the target location.

The medicaments of the present disclosure may be supplied to a target location from an exogenous source, or they may be made in vivo by cells in the target location or cells in the same organism as the target location.

Medicaments of the present disclosure may be in any physiologically appropriate formulation. They may be administered to an organism by injection, topically, by inhalation, orally or by any other effective means.

The same medicaments and methodologies described above may be used to suppress or inhibit pathological angiogenesis. For example, they may treat or prevent a condition occurring in the liver, kidney, lung, heart and pericardium, eye, skin, mouth, pancreas, gastrointestinal tract, brain, breast, bone marrow, bone, genitourinary, a tumor, or a wound.

### Methods of Treating an Angiogenesis-Related Disease

The present disclosure provides methods of treating an angiogenesis-related disease, e.g. retinal neovascular diseases, neovascular age-related macular degeneration, diabetic retinopathy, retinopathy of prematurity, or caners. The methods generally involve administering to a subject in need thereof an effective amount of alpha-enolase (enolase-1, ENO-1) antagonist. In another aspect, the present disclosure further provides a use of the ENO-1 antagonist in manufacturing a medicament for treating an angiogenesis-related disease.

In some embodiments, the ENO-1 antagonist used in the method may include, but not limited to:
(1) an anti-ENO-1 antibody or the binding fragment thereof; or
(2) nucleic acid of anti-ENO-1 antibody or the binding fragment thereof, which is delivered into cells and expressed as intracellular anti-ENO-1 antibody or the binding fragment thereof.

Embodiments of the present disclosure will be illustrated with the following specific examples. One skilled in the art would appreciate that these examples are for illustration only and that other modifications and variations are possible without departing from the scope of the present disclosure.

### EXAMPLES

Example 1. Preparation of anti-ENO-1 Antibody

In accordance with embodiments of the present disclosure, a general method for the generation of anti-ENO-1 antibodies includes obtaining a hybridoma producing a monoclonal antibody against ENO-1. Methods to produce monoclonal antibodies are known in the art and will not be elaborated here. Briefly, mice are challenged with antigen (ENO-1) with an appropriate adjuvant. Then, the spleen cells of the immunized mice were harvested and fused with hybridoma. Positive clones may be identified for their abilities to bind ENO-1 antigen, using any known methods, such as ELISA.

In an embodiment, anti-ENO-1 antibody of the present disclosure may be a mouse or humanized anti-ENO-1 antibody, or a scFv or Fab fragment thereof. An exemplary anti-ENO-1 antibody comprises a heavy-chain variable domain having three complementary regions including HCDR1 (GYTFTSCVMN; SEQ ID NO: 1), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6).

In accordance with embodiments of the present disclosure, the antibodies may be mouse antibodies. Alternatively, the antibodies may be chimeric antibodies (e.g., human constant regions coupled to the mouse variable regions) or humanized antibodies (e.g., mouse CDRs grafted on the framework regions of human immunoglobulins) or completely human antibodies.

The monoclonal antibody may be humanized by obtaining the CDR sequences from the hybridoma and cloning the CDR sequences into human framework sequences to produce humanized antibodies. Any common methods known in the art for identifying CDR sequences may be used. The CDR regions in this invention are identified with the Kabat number scheme. First, a hybridoma of anti-ENO-1 (e.g., mouse hybridoma) was generated. Such a hybridoma may be generated with standard protocols to produce monoclonal antibodies. The total RNA of the hybridoma was then isolated, for example using the TRIzol® reagent. Then, cDNA was synthesized from the total RNA, for example using a first strand cDNA synthesis kit (Superscript III) and an oligo (dT₂₀) primer or an Ig-3′ constant region primer.

Heavy and light chain variable regions of the immunoglobulin genes were then cloned from the cDNA. For example, the VH and VL variable regions of the anti-ENO-1 mAb were amplified from mouse hybridoma cDNAs by PCR, using a mouse Ig-5′ primer set (Novagen). The PCR products may be cloned directly into a suitable vector (e.g., a pJET1.2 vector) using CloneJet™ PCR Cloning Kit (Ferments). The pJET1.2 vector contains lethal insertions and will survive the selection conditions only when the desired gene is cloned into this lethal region. This facilitates the selection of recombinant colonies. Finally, the recombinant colonies were screened for the desired clones, the DNAs of those clones were isolated and sequenced. The immunoglobulin (IG) nucleotide sequences may be analyzed at the international ImMunoGeneTics information system (IGMT) website.

Antibody expression and purification

For antibody production, the isolated clones may be expressed in any suitable cells. As an example, F293 cells (Life technologies) were transfected with the anti-ENO-1 mAb expressing plasmid and cultured for 7 days. The anti-ENO-1 antibody was purified from the culture medium using a protein A affinity column (GE). Protein concentrations may be determined with a Bio-Rad protein assay kit and analyzed with 12% SDS-PAGE, using procedures known in the art or according to the manufacturer's instructions.

Example 2. In vitro effects of ENO-1 antagonist on VEGF-A-stimulated endothelial cells

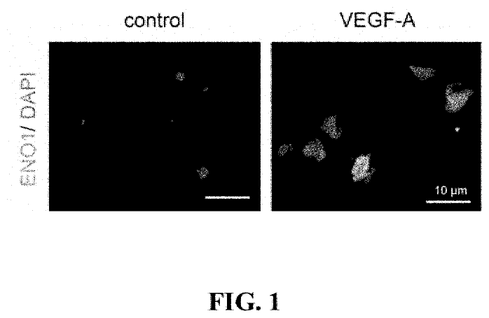

Primary human umbilical vein endothelial cells (HUVEC) grown on 4-well glass slides were treated with vascular endothelial growth factor A (VEGF-A) for 4 hours and fixed with fixation buffer (BioLegend) for 15 minutes at 4° C., blocked in 0.1% bovine serum albumin for 1 hour, and incubated with anti-ENO1 primary antibody (Abnova, Catalog No.: H00002023-M01) for 1 hour. After a brief wash with phosphate buffered saline (PBS), slides were incubated for 1 hour with Alexa Fluor 488 conjugated goat anti-mouse IgG secondary antibody (Invitrogen). After a brief wash with PBS, cell nuclei were stained with DAPI (Sigma) for 5-10 minutes and mounted with Fluoromount-G mounting medium (Southern Biotech). Images were acquired using a Nikon microscope. FIG. 1 showed the VEGF-A treatment increased surface expression of ENO-1 on HUVEC as examined by immunofluorescence staining.

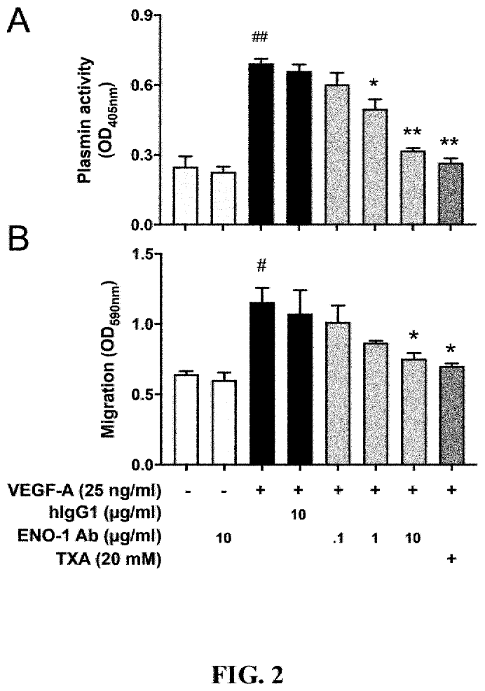

HUVEC cells stimulated with VEGF-A (PeproTech) for 4 hours were washed twice with PBS, resuspended in PBS at 10⁶ cells/ml, and preincubated with 30 μM of human Glu-plasminogen (Sigma) in the absence or presence of various concentration of ENO-1 antibody or control antibody human IgG1 at 37° C. for 1 hour. After incubation, the cells were washed with PBS for 3 times and resuspended in PBS followed by incubation with 1.5 nM of tissue plasminogen activator (Sigma) and 0.1mM of plasmin substrate Chromogenix S-2251 (Diapharma) at 37° C. for 2.5 hours. The plasmin activity was determined by measurement of the absorbance at 405 nm. FIG. 2A showed ENO-1 antibody dose-dependently reduced VEGF-A induced plasmin activation. TXA (tranexamic acid), a lysine analogue developed to saturate the binding sites of plasminogen and thus prevent plasmin formation, was used as positive control. For migration assay, after adding 900 μl of medium containing 10% fetal bovine serum (FBS) to the bottom well, the coating-free insert (Corning) was placed in the bottom well. HUVEC cells treated with VEGF-A for 4 hours were resuspended in medium supplemented with 2% FBS and seeded into the transwell in the absence or presence of various concentrations of ENO-1 antibody, control antibody human IgG1, or TXA. The cells were allowed to migrate at 37° C. for 18hours. The remaining cells on the upper part of the insert were removed by gently wiping with a cotton swab. The cells on the lower side of the insert were fixed with methanol for 10 minutes, followed by staining with 1% crystal violet for an additional 2 hours. The insert was gently washed with PBS and dried. The bound crystal violet was eluted with 33% acetic acid and the absorbance at 590 nm was measured. Since plasmin is known to regulate cell migration, FIG. 2B showed ENO-1 antibody dose-dependently reduced VEGF-A induced migration of HUVEC cells, which agreed with the results in FIG. 2A.

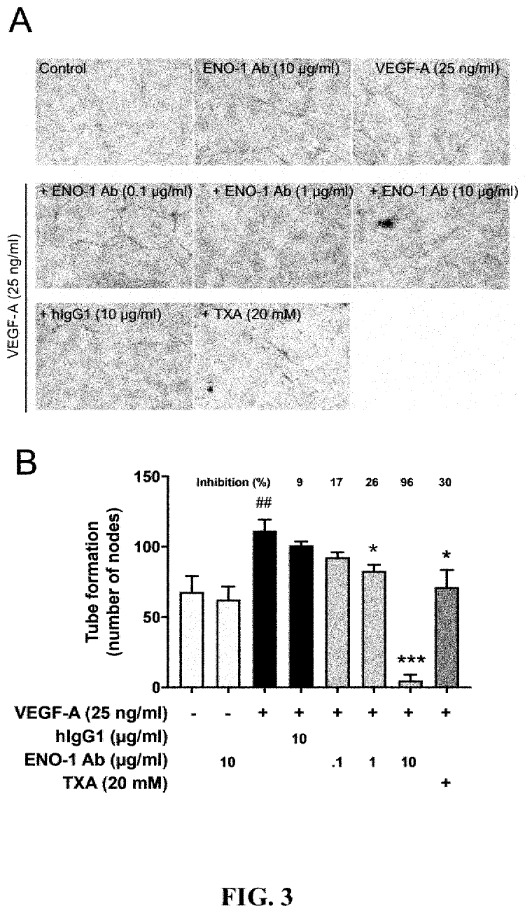

HUVEC cells grown on Matrigel-coated 96-well plate and were stimulated with VEGF-A for 24 hours in the absence or presence of various concentration of ENO-1 antibody, control human IgG1, or TXA. The formation of capillary-like tubes was observed and quantified as manual counting of nodes. FIG. 3 demonstrated the treatment of ENO-1 antibody dose-dependently reduced tube formation of VEGF-A-stimulated HUVEC. These results suggested in vitro anti-angiogenesis effects of ENO-1 antagonizing antibody on activated endothelial cells.

Example 3. Ex vivo effects of ENO-1 antagonist on VEGF-A-stimulated vessel sprouting.

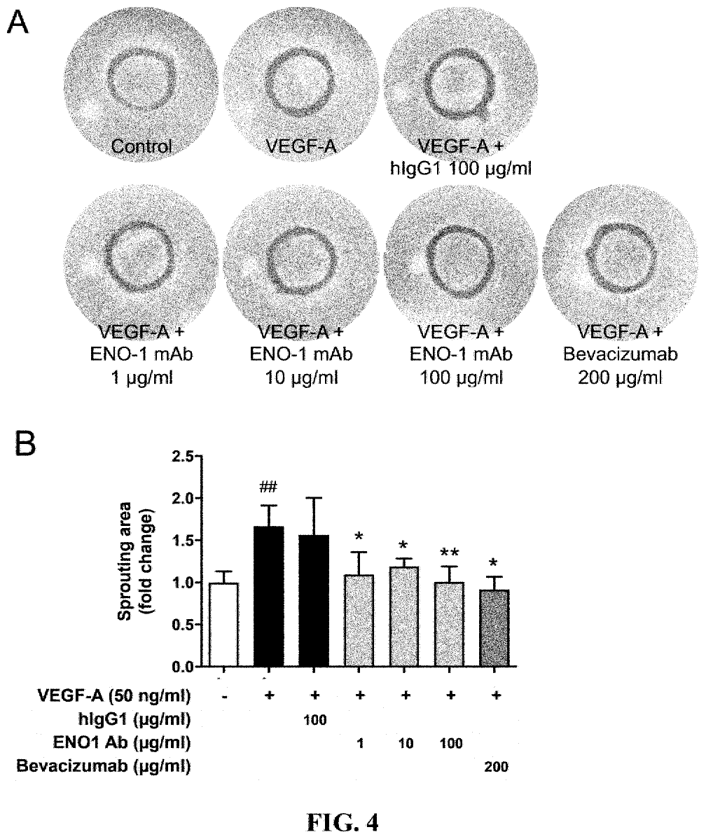

To examine the anti-angiogenesis effects of the ENO-1 antagonist, an ex vivo rat aortic ring sprouting assay was used. Isolated thoracic aortic ring segments (1 mm) immersed in Matrigel were treated with VEGF-A or VEGF-A combined with various doses of ENO-1 antibody for 4 days. Human IgGI (hlgG1) and VEGF monoclonal antibody (Bevacizumab) were used as negative and positive controls, respectively. As shown in FIG. 4, the sprouting microvessels were significantly increased by VEGF-A compared with the control group, especially inside the ring, a complex network was formed after VEGF-A treatment. The growth of new microvessels were both significantly inhibited in ENO-1 antibody or bevacizumab groups.

Example 4. In vivo anti-angiogenesis effects of ENO-1 antagonist.

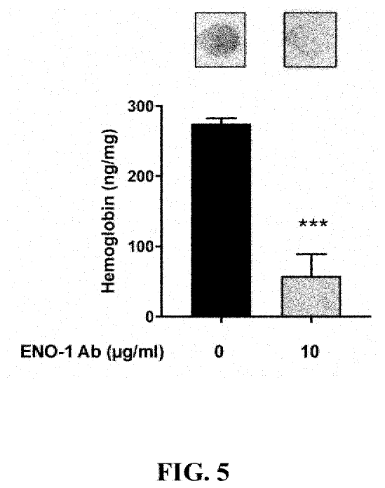

We used a tumor-cells-elicited angiogenesis Matrigel plug assay to access the anti-angiogenesis effects of ENO-1 antagonist in vivo. BALB/c nude mice (Lasco Co, Ltd.) were subcutaneously implanted with Matrigel mixed with RPMI 8226(BCRC No. 60384) human myeloma cells (1:1) with/without ENO-1 antibody into the right flanks of mice. After 7 days of treatment, the plugs were removed and the angiogenic response was quantified by the measurement of hemoglobin levels in the plugs by using Enzyme-linked immunosorbent assay (ELISA). Results in FIG. 5 showed ENO-1 antagonist significantly reduced the hemoglobin content of the plugs compared with the control group.

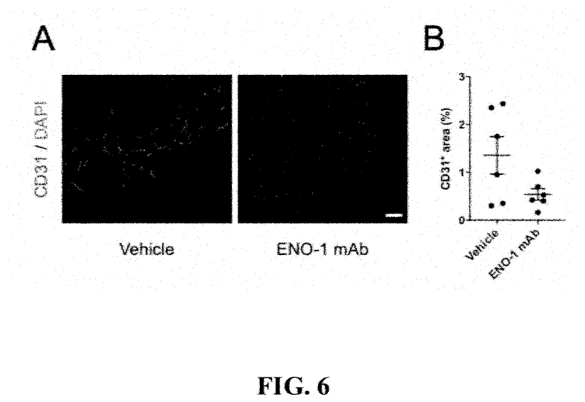

Androgen-independent human prostate cancer cell line PC-3 (ATCC No. CRL-1435) was used to establish a subcutaneous xenograft model in male BALB/c nude mice (Lasco Co, Ltd.). Before inoculation, PC-3 cells were washed with phosphate buffered saline (PBS) and resuspended with PBS and Matrigel at 1:1 for a final concentrations of 10⁷ cells/ml. Cells (10⁶ cells/100 μl) were implanted subcutaneously into the right flank of mice. Three days after implantation of PC-3cells, mice were randomized, and ENO-1 antibody (30 mg/kg) was dosed by intraperitoneal injection twice a week. At day 23, tumors were harvested for measurement of intratumoral angiogenesis by immunofluorescence staining of CD31expression. FIG. 6 showed a trend of reduced CD31 expression indicating an in vivo anti-angiogenesis activity of ENO-1 antagonist.

In summary, either in vitro or in vivo, ENO-1 antagonist significantly attenuated angiogenesis via reducing plasmin activity, cell migration, tube formation and vessel sprouting. Thus, ENO-1 antagonist is

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A method for treating an angiogenesis-related disease, comprising:
administering to a subject in need thereof an effective amount of alpha-enolase (enolase-1,ENO-1) antagonist.

2. The method of claim 1, wherein the ENO-1 antagonist is an anti-ENO-1 antibody or binding fragment thereof.

3. The method of claim 2, wherein the antibody is a monoclonal antibody.

4. The method of claim 2, wherein the antibody or binding fragment thereof comprises
a heavy-chain variable domain having three complementary regions including
HCDR1 (GYTFTSCVMN; SEQ ID NO: 1),
HCDR2 (YINPYNDGTKYNEKFKG; SEQ ID NO: 2), and
HCDR3 (EGFYYGNFDN; SEQ ID NO: 3); and
a light-chain variable domain having three complementary regions including
LCDR1 (RASENIYSYLT; SEQ ID NO: 4),
LCDR2 (NAKTLPE; SEQ ID NO: 5), and
LCDR3 (QHHYGTPYT; SEQ ID NO: 6).

5. The method of claim 2, wherein the antibody or binding fragment thereof is a mouse antibody, a human antibody, a chimeric antibody, a humanized antibody, or an antibody fragment thereof.

6. The method of claim 1, wherein the ENO-1 antagonist is nucleic acid to be delivered into cells and expressed as intracellular protein or peptide.

7. The method of claim 6, wherein the ENO-1 antagonist is secreted, non-secreted, or a combination thereof.

8. The method of claim 6, wherein the ENO-1 antagonist is delivered via a viral vector, a polymer, and/or liposome.

9. The method of claim 1, wherein the angiogenesis-related diseases comprise retinal neovascular diseases, neovascular age-related macular degeneration, diabetic retinopathy, retinopathy of prematurity, or caners.

10. canceled.
